(19) United States
(12) Patent Application Publication
(10) Pub. No.: US 2016/0019772 A1
(43) Pub. Date: Jan. 21, 2016

(54) INTEGRATED BREATHALYZER KIOSK AND ASSOCIATED METHODS OF USE

(71) Applicant: SAFELIFE.08 LLC, Sandy, UT (US)

(72) Inventors: Michael Chacon, Sandy, UT (US); Chris Anderson, Sandy, UT (US); Tori Riccardi, Sandy, UT (US)

(21) Appl. No.: 14/734,740

(22) Filed: Jun. 9, 2015

Related U.S. Application Data
(60) Provisional application No. 62/009,671, filed on Jun. 9, 2014.

(51) Int. Cl. G08B 21/18; G01N 33/497; H04W 4/00; G06Q 30/02; G06Q 40/08

(52) U.S. Cl. CPC G08B 21/182; G06Q 30/0268; G06Q 40/08; H04W 4/005; G01N 33/4972

(57) ABSTRACT

A breathalyzer kiosk can include a breath blood alcohol content sensor, a display, and a digital signage content module. The breath blood alcohol content sensor is capable of receiving breath of a patron and correlating detected alcohol levels within the breath to a blood alcohol content data of the patron. The display can be used to visually output a variety of information including a blood alcohol content value calculated from the blood alcohol content data provided by the alcohol sensor. The digital signage content module is also operatively connected to the display and is adapted to receive the blood alcohol content data and display a blood alcohol content value acquired from the blood alcohol content data. The digital signage content module also creates a multi-frame screen including a plurality of frames each having independent digital feeds, where at least one of the frames is an alcohol score report frame. The digital signage content module is also to display the score report frame without payment from the patron. Multiple breathalyzer kiosks can be linked via a content distributor module. A corresponding method of raising blood alcohol content awareness at an entertainment venue can also include orienting at least one breathalyzer kiosk within the entertainment venue at locations accessible to patrons.

### RELATED APPLICATION(S)

This application claims priority to U.S. Provisional Application No. 62/009,671 filed Jun. 9, 2014, which is incorporated herein by reference.

### GOVERNMENT INTEREST

None.

### BACKGROUND

A vast array of entertainment options are currently available for consumers to enjoy first hand experiences with a variety of engaging activities. Such entertainment options include, among others, sports, fairs, concerts, and other gatherings of people for purposes of entertainment. Entertainment venues frequently provide space for vendors to sell alcoholic beverages for consumption by patrons during such entertainment activities. Unfortunately, such alcohol consumption can result in impairment of judgment. Although a vast majority of such patrons consume alcohol responsibly, some individuals become intoxicated and can create a dangerous environment for themselves and others which lead to risk events. Such events can range from altercations, falls, minor injuries, and verbal assaults to drunk driving, dismemberment, permanent disability, and even death.

Current laws create potential legal liability of the entertainment venues for events which transpire as a result of alcohol sales originating on their property. Current approaches to mitigating such risks include advertising campaigns, pressuring vendors to limit consumption to certain individuals, Safe Fan Act regulation, and obtaining insurance. Although these efforts are somewhat effective, unfortunately such approaches provide only limited results in terms of consistency and reducing risk to patrons and the entertainment venue.

### SUMMARY

Recognizing that such limitations are undesirable, the inventors have developed an integrated breathalyzer kiosk which can be effectively utilized to increase awareness of blood alcohol content among patrons, while also reducing risk to patrons, other individuals, and entertainment venues. Accordingly, a breathalyzer kiosk can include a breath blood alcohol content sensor, a display, and a digital signage content module.

The breath blood alcohol content sensor can include a breath inlet fluidly connected to an alcohol sensor. The breath inlet is capable of receiving breath of a patron while the alcohol sensor is capable of correlating detected alcohol levels within the breath to a blood alcohol content data of the patron. The display can be used to visually output a variety of information including a blood alcohol content value calculated from the blood alcohol content data provided by the alcohol sensor.

The digital signage content module is also operatively connected to the display and is adapted to receive the blood alcohol content data and display a blood alcohol content value acquired from said blood alcohol content data. The digital signage content module is also configured to create a multi-frame screen and output the multi-frame screen to the display. Further, the multi-frame screen includes a plurality of frames each having independent digital feeds. However, at least one of said plurality of frames is an alcohol score report frame which includes the blood alcohol content value. At least one other of said plurality of frames is an informational frame which includes informational content (e.g. advertising, venue information, team information, news, etc). The digital signage content module is also configured to operate and display the score report frame without payment from the patron.

Although a single kiosk may be used, most entertainment venues are capable of hosting thousands or tens of thousands of patrons. As such, a breathalyzer kiosk system can be constructed which includes at least one breathalyzer kiosk (e.g. often more than three kiosks, and in many cases more than a dozen kiosks) and a content distributor module. The content distributor module is designed as a centralized data distribution node to communicate the plurality of independent digital feeds to each breathalyzer kiosk. Typically, each breathalyzer kiosk includes a wireless receiver adapted to receive the plurality of independent digital feeds from the content distributor module. The content distributor module can also be configured to synchronize digital feeds across each kiosk or can be designed to vary digital feeds for each kiosk independently.

An additional embodiment of the present invention includes a method of raising blood alcohol content awareness at an entertainment venue. This can involve orienting at least one breathalyzer kiosk within the entertainment venue at locations accessible to patrons. Advertising material from at least one participating advertiser can be used to form an advertising frame as at least one of the plurality of frames displayed on the breathalyzer kiosk. Such a method can also include obtaining reduced liability insurance rates for said entertainment venue.

The entertainment venue can often be a venue capable of hosting a large number of patrons along with a core entertainment event. Non-limiting examples of such entertainment venues can include a sports venue, a concert venue, a fair, and a conference center. Although any event which attracts large numbers of people at which alcohol is served can benefit from the systems and methods, sports events are of particular interest. Non-limiting examples of sports venues include a football stadium, a baseball stadium, a basketball stadium, a soccer stadium, an automotive race track, and an ice hockey arena.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a schematic block diagram of a breathalyzer kiosk system including a breathalyzer kiosk and a content distributor module in accordance with one embodiment of the invention.

FIG. 2 is a front view of a multi-feed display having a breathalyzer inlet in accordance with another embodiment of the invention.

FIG. 3 is a schematic diagram of a breathalyzer kiosk system including n breathalyzer kiosks in accordance with yet another embodiment of the invention.

FIG. 4 is a block diagram of a method of using such systems to raise blood alcohol content awareness at an entertainment venue in accordance with another embodiment of the invention.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a feed” includes reference to one or more of such signals and reference to “displaying” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Breathalyzer Kiosks

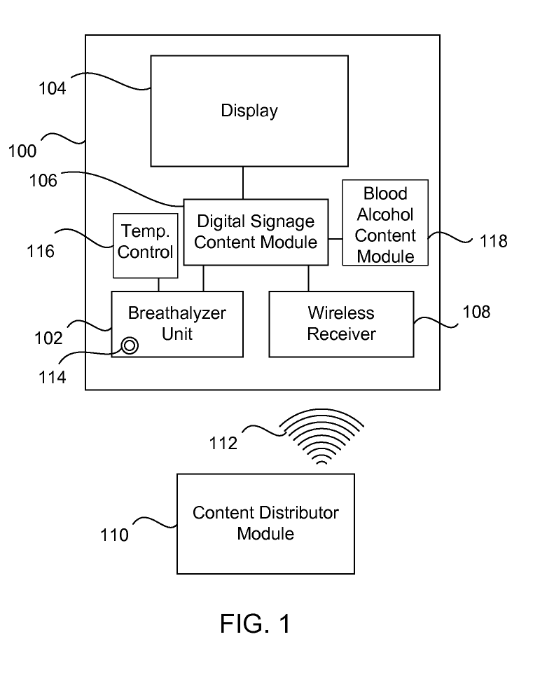

Referring to FIG. 1, a breathalyzer kiosk 100 can include a breath blood alcohol content sensor 102, a display 104, and a digital signage content module 106. The digital signage content module generally receives digital feeds from an informational source and the blood alcohol sensor to form a visual output including multiple frames. An optional wireless receiver 108 can be connected to the digital signage content module. The wireless receiver can communicate with a remote content distributor module 110 as the informational source via a wireless signal 112. One of the frames includes a blood alcohol content value obtained from a breath of a patron. The blood alcohol content value is also obtained and displayed without charging the patron or obtaining personal information of the patron.

The breath blood alcohol content sensor 102 can calculate a blood alcohol content from a patron's breath. The breath blood alcohol sensor can include a breath inlet fluidly connected to an alcohol sensor. The breath inlet is capable of receiving breath of a patron and the alcohol sensor is capable of correlating detected alcohol levels within the breath collected by the breath inlet to a blood alcohol content data of the patron. Correlation can be accomplished through use of any known method used for such purposes. The breath inlet can be an orifice 114 into which a replaceable straw or other conduit can be placed.

The breath blood alcohol content sensor can be any suitable sensor which is capable of determining blood alcohol content of a patron. Current sensor technology is limited to breath based measurement, however other sensors may be used if blood alcohol content can be determined non-invasively. Suitable breath blood alcohol sensors can be a fuel cell based sensor, an infrared based sensor, or a semiconductor based sensor. Although each sensor type has benefits, fuel cell based sensors are currently particularly useful and reliable. Platinum based fuel cell sensors can be used such as those available from Lifeloc Technologies (FC series: FC10, FC10Plus, FC20, and FC20BT, Lifeguard, and Workplace series). Other suitable breath blood alcohol sensors can include, but are not limited to, KHN Solutions Inc. dba BackTrack, AK Solutions USA LLC (AlcoMate® series and AlcoHawk® series), and Canadian Breath Analyzer Corp. Many of these sensors are also currently DOT and NHSTA approved. Integrating a DOT and/or NHSTA approved breathalyzer unit can provide benefits of added reliability and improved ability to convince patrons and insurance carriers regarding the value of these breathalyzer kiosk systems.

The breath blood alcohol sensor can optionally further include a calibration mode such that the breath alcohol content sensor can be recalibrated. Sensor drift and loss of accuracy can occur over time. The rate of sensor drift can depend somewhat on operating conditions such as service time, temperature, frequency of test cycles, humidity, ambient particulate count, and sensor hardware, among other factors. Recalibration can be accomplished using physical cleaning of the sensor and/or adjustments to software baseline settings. For example, recalibration can use a dry gas approach or a wet bath approach to realign the sensor signal with a known reference signal. As a general guideline recalibration can occur at least once a year, although high use or other factors can reduce recommended recalibration times to less than six months and in some cases monthly.

Although recalibration can assist in preserving accuracy under a given set of conditions, such calibration is set against specific temperature conditions. Temperature extremes which exceed the calibrated temperature can result in inaccurate readings since the correlation accuracy can also be a function of temperature. Accordingly, in order to keep the sensor within accurate operating limits, an optional temperature control unit 116 operatively can be thermally associated with the breath blood alcohol content sensor 102 to maintain a temperature of the sensor within a target operational temperature range. The temperature control unit can include at least one of a heating mechanism or a cooling mechanism, although both heating and cooling mechanisms can be used together. Such temperature control units can include fans, electrical resistive heaters, heat transfer fins, refrigerant systems, and the like. Although the target operational temperature range can vary depending on the sensor configuration, temperatures from −10° C. to 45° C. can generally be suitable. In some cases a target operational temperature range can be 0° C. to 40° C. and in other cases from 10° C. to 30° C.

The display can be used to visually output a variety of information including a blood alcohol content value calculated from the blood alcohol content data provided by the alcohol sensor. Suitable displays can generally be LCD or LED based electronic displays, although any display which is capable of creating a visible image from a data stream can be used. More specifically, the digital display is configured to receive and output the multi-frame screen as a coherent visual image. In one alternative, the display can be a touch responsive screen which is adapted to receive input from the patron. In this manner various features can be customized for the patron. For example, breathalyzer instructions, recommendations, or requests can be reviewed by the patron. In addition, as explained more fully below, some frames may include advertising such that a patron can touch specific frame locations in order to obtain additional information about a specific product offering.

The digital signage content module is also operatively connected to the display and is adapted to receive the blood alcohol content data and display a blood alcohol content value acquired from said blood alcohol content data. The digital signage content module is also configured to create a multi-frame screen and output the multi-frame screen to the display. Further, the multi-frame screen includes a plurality of frames each having independent digital feeds. However, at least one of said plurality of frames is an alcohol score report frame which includes the blood alcohol content value. At least one other of said plurality of frames is an informational frame which includes informational content (e.g. advertising, venue information, team information, news, etc).

The digital signage content module is also configured to operate and display the score report frame without payment from the patron. For example, the breathalyzer can be actuated directly without a payment authorization or other data input from the patron. A breath test cycle can be initiated simply by breathing into the unit, or by pressing a corresponding test cycle start feature (e.g. a touch screen icon, a manual button integrated into a housing of the kiosk, a button on an outer frame of the display, etc). When this interaction occurs, message codes within the breathalyzer module are transmitted via an output port adaptor to the digital signage content module.

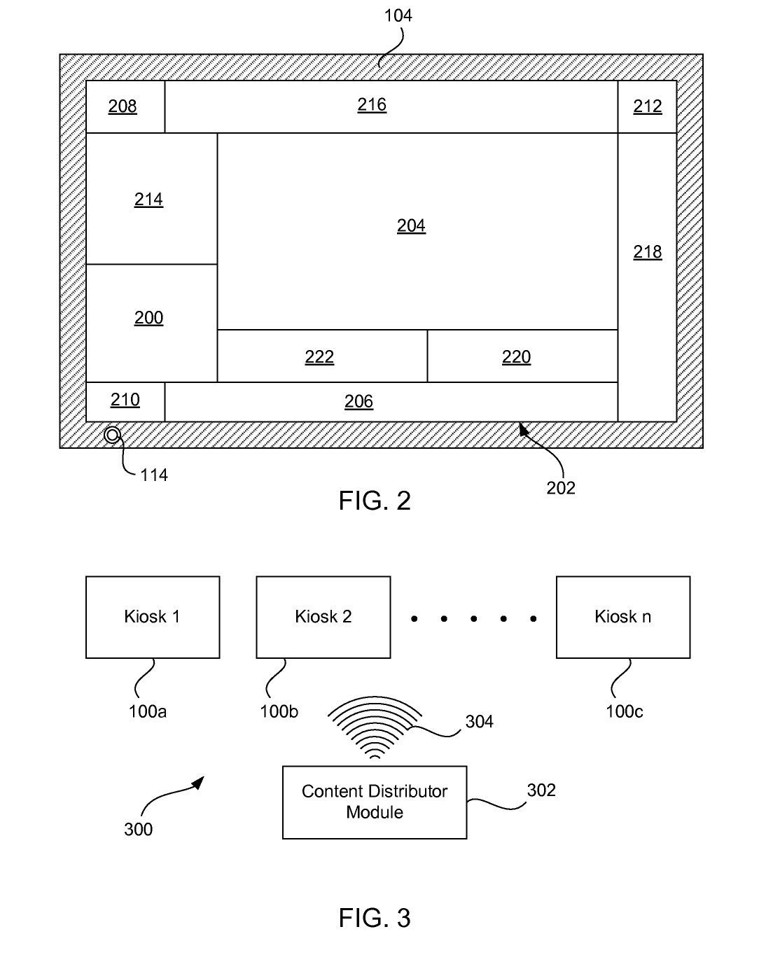

Upon initiation of a test cycle, a patron blows into the breathalyzer unit at which point the breath blood alcohol content sensor analyzes an alcohol content of the breath. A corresponding signal is generated by the breath blood alcohol content sensor as a blood alcohol content data. This blood alcohol content data is then communicated to the digital signage content module. The digital signage content module then forms a blood alcohol content value, using any accepted algorithm or other method used for such purposes, which can be integrated into the score report frame 200 which is displayed as part of the multi-frame screen 202 as shown in FIG. 2. The score report frame includes the blood alcohol content value. In an optional embodiment, an alert can be provided when the value exceeds a predetermined legal and/or health limit. For example, a visible warning can be triggered when the value meets or exceeds 0.05, while additional visible and/or audible warnings may be triggered at values in excess of 0.15 or 0.2, for example. Upon completion of the test cycle the score report frame is displayed for a limited time. Typically, the score report frame is then reset and the data is not stored. The limited time can range from about 3 seconds to about 1 minute, and typically less than about 10 seconds. Optionally, the data can be cached in storage and/or transmitted for further analysis. For example, data can be stored in order to assess blood alcohol levels as a function of various locations, differing events, seasons, days of the week, time of the event, and the like.

Referring again to FIG. 2 the display 104 can include the plurality of frames within the multi-frame screen 202, which can be arranged in almost any visually acceptable configuration. Although different configurations are suitable, FIG. 2 illustrates an arrangement which includes a core video frame 204, the score report frame 200, a running ticker frame 206, a team branding frame 208, a league branding frame 210, a time frame 212, and a plurality of advertising frames 214, 216, 218, 220, and 222, each advertising frame having an independent data feed. Generally, a variety of frames can be utilized to communicate information such as, but not limited to, a video frame, a league branding frame, a team branding frame, a time frame, a live news feed frame, an external advertising frame, an in-venue vendor advertising frame, a weather forecast frame, a breathalyzer instructions frame, a legal disclaimer frame, a venue announcement frame, an alert frame (e.g. stadium, emergency or public safety alerts), and combinations thereof. Furthermore, such multi-frame screens can be dynamically changed and adjusted. In some cases, the plurality of frames includes at least one video frame. Optionally, at least one of the plurality of frames includes a legal disclaimer regarding the breathalyzer test.

The digital signage module can also further include a blood alcohol content module 118, as shown in FIG. 1, adapted to receive the blood alcohol content data and calculate the blood alcohol content value. This blood alcohol content module can include at least one of an input port adapter and a software image module. The input port adapter is a connector which receives a signal (e.g. usually a wired signal such as a USB port or other I/O port, but a wireless signal can also be used) from the breath blood alcohol content sensor. The software image module can include coding to integrate the blood alcohol content data into a blood alcohol content value which can be formed into the score report frame by the digital signage module.

The interface between the digital signage content module and the blood alcohol content module operates such that machine codes are interpreted based on the “simple” data codes produced by the blood alcohol content module which are modified into a streaming digital signage output. The basic codes indicate the next steps to take in order to successfully produce blood alcohol content results. These prompts may include, “machine not ready”, “please blow steadily for at least 4 seconds”, “test complete”, “your test score is”, etc. These codes can be either passed through to the screen output as part of one or more frames, or actually interpreted and used by the digital signage content module to run specific graphics files, advertisements, video, etc. Thus, when data output is received by the digital signage module from the blood alcohol content module, they are then processed and encoded to produce a streaming content and output in a variety of graphic formats as part of the score report frame or other frames to the digital signage portion of the screen correlated for that purpose.

The independent digital feeds which are used to produce the multi-frame screen can be pre-installed on the digital signage module or such information can be dynamically uploaded via wired or wireless connections with a remote content distributor module 110 as shown in FIG. 1. Accordingly, the digital signage module 106 can optionally comprise a wireless receiver 108 adapted to receive the independent digital feeds other than the blood alcohol content, from the remote content distributor module 110.

The breathalyzer kiosk can be configured in a variety of different form factors. For example, a standalone kiosk can be provided with a housing which includes a base. The housing secures the display at a convenient height and protects components from environmental exposure and tampering. The display height can generally be between about 3 feet and 5.5 feet for a standing configuration, or about 2 feet to about 3.5 feet for a sitting configuration. Alternatively, the breathalyzer kiosk can be configured as a mountable unit which can be mounted on a wall, fence, post, or other structure. In such cases, the components of the kiosk can be housed within a housing which includes access openings, mounting elements, data connections, and/or power connections. Additionally, the breathalyzer kiosk can be configured as a plug-in device to access common AC outlet current, although batteries can also be used to provide portability or back-up power.

Although a single kiosk may be used, most entertainment venues are capable of hosting thousands or tens of thousands of patrons. As such, a breathalyzer kiosk system 300 can be constructed which includes at least one breathalyzer kiosk 100a and a content distributor module 302 as shown in FIG. 3. Frequently, more than three kiosks can be used, and in many cases more than a dozen kiosks can be used. As a generalization, n kiosks can be part of the system which includes a first kiosk 100a, and second kiosk 100b, and additional kiosks up to and including an n^{th} kiosk 100c. A small event venue may include two or three kiosks, while a larger venue may utilize several dozen kiosks (e.g. up to 100 or more).

The content distributor module 302 is designed as a centralized data distribution node to communicate the plurality of independent digital feeds to each breathalyzer kiosk 100a to 100c. Typically, each breathalyzer kiosk includes a wireless receiver adapted to receive the plurality of independent digital feeds from the content distributor module via a wireless signal 304. Each breathalyzer kiosk can be oriented at strategic locations throughout the venue, e.g. near concessions, bathrooms, exits, or other locations unique to a specific venue. The content distributor module can also be configured to synchronize digital feeds across each kiosk or can be designed to vary digital feeds for each kiosk independently.

The content distributor module can include a signal generator to send the plurality of independent digital feeds. As mentioned, such signals can be relayed via wired or wireless protocols. As such, an Ethernet connection, phone connection, optical fiber, or other data relay can be used. Many types of wireless devices are capable of communicating via a licensed spectrum, such as through a cellular network, and via unlicensed spectrum, such as via a WiFi hotspot. In cases of using a cellular network, the wireless receiver can include a subscriber identity module (SIM card) for connection via an existing cellular network. Alternatively, WiFi receivers can be used as the wireless receiver. WiFi is a common name provided to an Institute of Electronics and Electrical Engineers (IEEE) 802.11 set of standards for communicating in unlicensed spectrum including the 2.4, 3.7 and 5 GHz frequency bands. The set of standards includes the IEEE 802.11a standard released in 1999 for communication in the 5 GHz and 3.7 GHz band, the IEEE 802.11b standard, also released in 1999 for communication in the 2.4 GHz band, the 802.11g standard released in 2003 for communication in the 2.4 GHz range via orthogonal frequency division multiplexing (OFDM) and/or direct sequence spread spectrum (DSSS), and the 802.11n standard released in 2009 for communication in the 2.4 GHz and 5 GHz bands using multiple-input multiple-output (MIMO).

Standards such as WiFi or Bluetooth are used to provide wireless local area networks (WLAN) that can be accessed by dual mode devices that are also capable of accessing a cellular networking standard such as IEEE 802.16 standard, commonly referred to as WiMAX (worldwide interoperability for microwave access), and the third generation partnership project (3GPP). Releases of the IEEE 802.16 standard include the IEEE 802.16e-2005, 802.16-2009, and 802.16m-2011. Releases of the 3GPP standard include the 3GPP LTE, Release 8 in the fourth quarter of 2008 and 3GPP LTE Advanced Release 10 in the first quarter of 2011.

The system described herein can also provide a mechanism to wirelessly communicate with patrons directly via their mobile devices. In one alternative, the system can include a wireless signal generator configured to send and receive digital content with a mobile device of the patron. The wireless signal generator can be integrated as part of the content distributor module and/or one or more of the breathalyzer kiosks. Depending on signal coverage, it can be desirable to orient such a wireless signal generator within the breathalyzer kiosks. In this manner the system can be configured to provide patrons with a desired venue wireless content. The venue wireless content can include, but is not limited to, Internet access, venue content, advertising, patron participation programs, or other content. For example, customized venue activities, announcements, or other programs can be communicated to mobile devices of patrons via the system. As such, the breathalyzer system can also benefit patrons who do not use the breathalyzer kiosk for blood alcohol content testing. Such wireless capability also provides the entertainment venue additional options to engage patrons with venue events, supplemental programs, advertising, or other useful content offerings. Wireless access in this manner can be unsecured or secured depending on the venue wireless content.

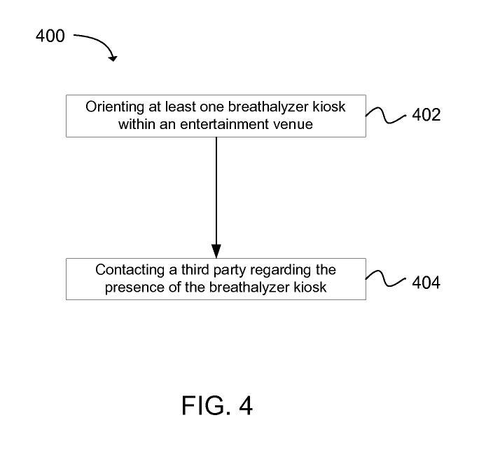

Referring now to FIG. 4, an additional embodiment of the present invention includes a method 400 of raising blood alcohol content awareness at an entertainment venue. This can involve orienting 402 at least one breathalyzer kiosk within the entertainment venue at locations accessible to patrons. The method can further include contacting 404 at least one third party regarding the presence of the breathalyzer kiosk. In some cases, the third party can be a participating advertiser. The participating advertiser can supply advertising material for integration into an advertising frame as part of the multi-frame screen. Such advertising can be for local businesses, national businesses, or even businesses located within the entertainment venue. Further, a third party can include an insurance carrier. The entertainment venue or other responsible party who holds liability insurance over the entertainment venue can contact the insurance carrier to provide notification of reduced risks associated with the presence and use of the breathalyzer kiosks described herein. As such, the method can also include obtaining reduced liability insurance rates for the entertainment venue. The method can also include communicating a message to the patrons regarding availability of the breathalyzer kiosk(s). This can be accomplished via audible announcements, notices printed on venue literature or posted in viewable locations throughout the venue, electronic notices to mobile devices, community programs, or otherwise communicated to patrons.

The entertainment venue can often be a venue capable of hosting a large number of patrons along with a core entertainment event. Non-limiting examples of such entertainment venues can include a sports venue, a concert venue, a fair, a circus, network reality TV, and a conference center. Although any event which attracts large numbers of people at which alcohol is served can benefit from the systems and methods, sports events are of particular interest. Non-limiting examples of sports venues include a football stadium, a baseball stadium, a basketball stadium, a soccer stadium, an automotive race track, an ice hockey arena, monster truck event, motocross event, and the like.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A breathalyzer kiosk, comprising:
a) a breath blood alcohol content sensor including a breath inlet fluidly connected to an alcohol sensor, said breath inlet capable of receiving breath of a patron and said alcohol sensor capable of correlating detected alcohol levels within the breath with a blood alcohol content data of the patron;
b) a display; and
c) a digital signage content module operatively connected to the display and adapted to receive the blood alcohol content data and display a blood alcohol content value from said blood alcohol content data, said digital signage content module being configured to create a multi-frame screen and output said multi-frame screen to the display, wherein the multi-frame screen includes a plurality of frames each having independent digital feeds, wherein at least one of said plurality of frames is a score report frame which includes the blood alcohol content value, and wherein at least one of said plurality of frames is an informational frame which includes informational content, said digital signage content module being configured to operate and display the score report frame without payment from the patron.

2. The kiosk of claim 1, wherein the breath blood alcohol content sensor is a fuel cell based sensor, an infrared based sensor, or a semiconductor based sensor.

3. The kiosk of claim 1, wherein the breath inlet is an orifice which is coupleable with a replaceable straw.

4. The kiosk of claim 1, further comprising a calibration mode wherein the breath alcohol content sensor can be recalibrated.

5. The kiosk of claim 1, wherein the display is a digital display configured to receive the multi-frame screen.

6. The kiosk of claim 1, wherein the display is a touch responsive screen adapted to receive input from the patron.

7. The kiosk of claim 1, wherein the digital signage module further comprises a blood alcohol content module adapted to receive the blood alcohol content data and calculate the blood alcohol content value.

8. The kiosk of claim 1, wherein the digital signage module further comprises a wireless receiver adapted to receive the independent digital feeds other than the blood alcohol content, from a remote content distributor module.

9. The kiosk of claim 1, wherein the score report frame includes an alert corresponding to incapacity limits.

10. The kiosk of claim 1, wherein the plurality of frames further includes a member selected from the group consisting of a video frame, a league branding frame, a team branding frame, a time frame, a live news feed frame, an external advertising frame, an in-venue vendor advertising frame, a weather forecast frame, a breathalyzer instructions frame, a legal disclaimer frame, an alert frame, and combinations thereof.

11. The kiosk of claim 1, wherein at least one of the plurality of frames includes a legal disclaimer regarding the breathalyzer test.

12. The kiosk of claim 1, further comprising a temperature control unit operatively associated with the breath blood alcohol content sensor to maintain a temperature of the sensor within a target operational temperature range.

13. A breathalyzer kiosk system, comprising:
a) at least one breathalyzer kiosk as in claim 1; and
b) a content distributor module configured to provide a plurality of the independent digital feeds to the at least one breathalyzer kiosk.

14. The system of claim 13, wherein the at least one breathalyzer kiosk includes a plurality of breathalyzer kiosks, each further comprising a wireless receiver adapted to receive the plurality of independent digital feeds from the content distributor module.

15. The system of claim 13, wherein the content distributor module includes a wireless signal generator to send the plurality of independent digital feeds.

16. The system of claim 13, further comprising a wireless signal generator configured to send and receive digital content with a mobile device of the patron.

17. A method of raising blood alcohol content awareness at an entertainment venue, comprising:
a) orienting at least one breathalyzer kiosk as in claim 1 within the entertainment venue at locations accessible to patrons.

18. The method of claim 17, further comprising including advertising material from at least one participating advertiser to form an advertising frame as at least one of the plurality of frames.

19. The method of claim 17, further comprising contacting an insurance carrier to obtain reduced liability insurance rates for said entertainment venue.

20. The method of claim 17, further comprising communicating a message to the patrons regarding availability of said at least one breathalyzer kiosk.
